(19) United States
(12) Patent Application Publication
(10) Pub. No.: US 2019/0053795 A1
(43) Pub. Date: Feb. 21, 2019

(54) SHAPE-MEMORY SUTURE

(71) Applicant: ASFORA IP, LLC, Sioux Falls, SD (US)

(72) Inventors: Wilson Theophilo Asfora, Sioux Falls, SD (US); Michael Edward Villalta, Yerington, NV (US)

(21) Appl. No.: 16/104,667

(22) Filed: Aug. 17, 2018

Related U.S. Application Data
(60) Provisional application No. 62/546,729, filed on Aug. 17, 2017.

(51) Int. Cl. A61B 17/04; A61B 17/11

(52) U.S. Cl. CPC A61B 17/0487; A61B 2017/00867; A61B 17/11

(57) ABSTRACT

The disclosure is related to a suture that is configured to connect two or more tissue portions. The suture may include shape-memory material. For example, the suture may be reconfigurable between loaded and relaxed states; when in the loaded state, the suture may be open, such that at least one end of the suture may be positioned adjacent to at least one tissue portion of the portions that are intended for connection by the suture.

### CROSS-REFERENCE TO RELATED APPLICATIONS

This application claims priority to U.S. Provisional Patent Application No. 62/546,729, filed Aug. 17, 2017, the contents of which are hereby incorporated by reference in their entirety.

### TECHNICAL FIELD

This disclosure is in the field of medical devices. More specifically, this disclosure is in the field of tissue repair.

### BACKGROUND

Various types of sutures and other devices are known for attaching body tissues together to facilitate the healing process. In certain procedures, such as in performing vascular anastomosis, the surgeon requires precision and manipulability to properly place and secure the suture while controlling the vessels to be connected. A locking suture that is easily used with precision is desired.

### SUMMARY

Embodiments disclosed herein generally relate to a suture that is configured to connect together two or more tissue portions. In some embodiments, the suture may include shape-memory material. For example, the suture may be reconfigurable between loaded and relaxed states; when in the loaded state, the suture may be open, such that at least one end of the suture may be positioned adjacent to at least one tissue portion of the portions that are intended for connection by the suture. When released from the loaded state, the suture may (under shape-memory material biasing) bend or twist toward the closed position (e.g., toward the relaxed state).

In one example, a suture includes a connector, a clasp, and a body. The connector includes a neck and a head. The head has a tip and a base, which has a width that is wider than the neck in a transverse direction. The head tapers from the base to the tip such that the head is configured to pierce animal tissue. The clasp includes sheet material that defines an opening that, in a transverse direction, is narrower than the width of the base of the head. The clasp is resilient to allow the opening to elastically expand in the transverse direction upon passing the head therethrough and contract about the neck to secure the connector to the clasp. The body, made of a shape-memory material, connects the connector to the clasp.

In one example, when the suture is in a relaxed state, the body and a portion of the neck are positioned on one side of the clasp and the head is positioned on an opposite side of the neck such that the head is secured to the clasp.

In another example, when the suture is in a relaxed state, the body generally aligns the tip with the clasp opening.

In a further example, when the suture is in a relaxed state, the tip is positioned adjacent to the clasp.

In one example, a suture includes a connector, a clasp, and a body. The connector includes a neck and a head. The head has a tip and a base, which has a width that is wider than the neck in a transverse direction. The head tapers from the base to the tip such that the head is configured to pierce animal tissue. The clasp includes sheet material that defines an opening that, in a transverse direction, is narrower than the width of the base of the head. The clasp is resilient to allow the opening to elastically expand in the transverse direction upon passing the head therethrough and contract about the neck to secure the connector to the clasp. The body, made of a shape-memory material, connects the connector to the clasp. In a relaxed state the body and a portion of the neck are positioned on one side of the clasp, and the head is positioned on an opposite side such that the head is locked to the clasp.

In one example, a suture includes a connector, a clasp, and a body. The connector includes a neck and a head. The head has a tip and a base, which has a width that is wider than the neck in a transverse direction. The head tapers from the base to the tip such that the head is configured to pierce animal tissue. The clasp includes sheet material that defines an opening that, in a transverse direction, is narrower than the width of the base of the head. The clasp is resilient to allow the opening to elastically expand in the transverse direction upon passing the head therethrough and contract about the neck to secure the connector to the clasp. The body, made of a shape-memory material, connects the connector to the clasp. In a relaxed state, the body generally aligns the tip with the clasp opening.

In one example, a suture includes a connector, a clasp, and a body. The connector includes a neck and a head. The head has a tip and a base, which has a width that is wider than the neck in a transverse direction. The head tapers from the base to the tip such that the head is configured to pierce animal tissue. The clasp includes sheet material that defines an opening that, in a transverse direction, is narrower than the width of the base of the head. The clasp is resilient to allow the opening to elastically expand in the transverse direction upon passing the head therethrough and contract about the neck to secure the connector to the clasp. The body, made of a shape-memory material, connects the connector to the clasp. In a relaxed state, the body positions the tip adjacent to the clasp.

In some examples, the head of any of the foregoing connectors has at least one curved surface or more than two sides tapering from the base to the tip.

In some examples, the body of any of the foregoing sutures includes shoulders that extend outward from opposites sides of the neck in a transverse direction. The shoulders may be positioned generally perpendicularly to a longitudinal axis of the neck and are configured to prevent penetration of the suture into tissue past the neck of the connector. A fillet may be positioned between each of the opposite sides of the neck and the respective shoulder. A fillet may be positioned between each shoulder and a respective lateral edge of the body.

In some example, any of the foregoing openings is partially defined by longitudinal walls having notches formed therein.

In one example, a suture includes a connector, a clasp, one or more edge-stops, and a body. The connector includes a neck and a head connected to the neck. The neck includes a tip and a base, which has a width that is wider than the neck in a transverse direction and tapers from the base to the tip such that the head is configured to pierce animal tissue. The clasp includes sheet material that defines an opening that is sized and configured to secure the connector to the clasp. The one or more edge-stops connect to the neck and are spaced from the base of the head. The one or more edge-stops together with the base of the head define a tissue-capture portion of the neck. The body, which is made of a shape-memory material, connects the connector to the clasp.

In one example, when the suture is in a relaxed state, the body and a portion of the neck are positioned on one side of the clasp and the head is positioned on an opposite side of the neck such that the head is secured to the clasp.

In another example, when the suture is in a relaxed state, the body generally aligns the tip with the clasp opening.

In a further example, when the suture is in a relaxed state, the tip is positioned adjacent to the clasp.

In some examples, the edge-stops are positioned generally perpendicularly to a longitudinal axis of the neck and are configured to prevent penetration of the suture into tissue past the neck of the connector.

In some examples, the head has at least one curved surface or more than two sides tapering from the base to the tip.

In some examples, the body includes shoulders that extend outward from opposites sides of the neck in a transverse direction. The shoulders may be positioned generally perpendicularly to a longitudinal axis of the neck and are configured to prevent penetration of the suture into tissue past the neck of the connector. A fillet may be positioned between each of the opposite sides of the neck and the respective shoulder. A fillet may be positioned between each shoulder and a respective lateral edge of the body.

In some example, the opening is partially defined by longitudinal walls having notches formed therein.

In one example, a method of connecting two or more tissue portions includes positioning a suture adjacent to at least one of the two or more tissue portions and releasing the suture from a loaded state, and thereby piercing the two or more tissue portions with a piercing tip of a connector of the suture. When positioned, the suture is in the loaded state and includes the connector, a clasp, and a body connecting the connector to the clasp. The connector includes a neck and a head. The head has a tip and a base, which has a width that is wider than the neck in a transverse direction, and the head tapers from the base to the tip such that the head is configured to pierce animal tissue. The clasp includes sheet material that defines an opening that, in a transverse direction, is narrower than the width of the base of the head. The clasp is resilient to allow the opening to elastically expand in the transverse direction upon passing the head therethrough and contract about the neck to secure the connector to the clasp.

In one example, releasing the suture from the loaded state positions the connector adjacent to the clasp.

In another example, releasing the suture from the loaded state secures the connector to the clasp.

In some examples, the method also includes securing the connector to the clasp.

In some examples, the body includes shoulders that extend outward from opposites sides of the neck in a transverse direction. After releasing the suture from the loaded state, the suture is positioned such that the tissue portions are positioned between the shoulders of the body and the base of the connector.

In one example, a method of connecting two or more tissue portions includes positioning a suture adjacent to at least one of the two or more tissue portions. The suture is in a loaded state and includes a connector, a clasp, and a body, which connects the connector to the clasp. The connector includes a neck and a head. The head has a tip and a base, which has a width that is wider than the neck in a transverse direction. The head tapers from the base to the tip such that the head is configured to pierce animal tissue. The clasp includes sheet material that defines an opening that, in a transverse direction, is narrower than the width of the base of the head. The clasp is resilient to allow the opening to elastically expand in the transverse direction upon passing the head therethrough and contract about the neck to lock the connector to the clasp. The method also includes positioning the connector adjacent to the clasp by releasing the suture from the loaded state and thereby piercing the two or more tissue portions with the piercing tip of the connector.

In another example, releasing the suture from the loaded state secures the connector to the clasp.

In some examples, the method also includes securing the connector to the clasp.

In some examples, the body includes shoulders that extend outward from opposites sides of the neck in a transverse direction. After releasing the suture from the loaded state, the suture is positioned such that the tissue portions are positioned between the shoulders of the body and the base of the connector.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments, wherein identical reference numerals refer to identical or similar elements or features in different views or embodiments shown in the drawings.

FIG. 1A is a perspective view of a suture in a flat condition according to an embodiment;

FIGS. 1B and 1C are top and side views, respectively, thereof;

FIGS. 2A and 2B are side, perspective views of the suture of FIG. 1A in a closed condition;

FIG. 3 is a perspective view of the suture of FIG. 1A in a pre-closed condition; and

FIGS. 4 and 5 are cutaway views of the suture of FIG. 1A connecting together two tissue portions according to an embodiment.

### DETAILED DESCRIPTION

Embodiments disclosed herein generally relate to a suture that is configured to connect together two or more tissue portions. In some embodiments, the suture may include shape-memory material, spring metal, or other suitable materials. For example, the suture may be reconfigurable between loaded and relaxed states; when in the loaded state, the suture may be open or unlocked, such that at least one end of the suture may be positioned adjacent to at least one tissue portion of the portions that are intended for connection by the suture. When released from the loaded or open state, the suture may (under shape-memory material biasing) bend or twist toward the closed position (e.g., toward the relaxed state).

For example, as the suture is reconfigured toward its relaxed state (or the parent shape of the shape-memory material), at least one portion of the suture may penetrate one or more of the tissue portions. In an embodiment, the suture may include a head that may be sized and configured to penetrate the tissue portions as the suture is transformed from the loaded state toward or to the relaxed state. In some embodiments, the stiffness of the suture material and/or the shape of the head may be configured in a manner that the suture can penetrate tissue under its own bias toward its relaxed state. For example, the head may be generally arrow-shaped and have a suitable tip or point that may be configured to penetrate the tissue portions.

In some embodiments, the suture includes a clasp and a connector. For example, the suture may be passed through the tissue portions, such that the connector of the suture is positioned adjacent to the clasp. Moreover, the clasp and the connector may be sized and configure such that the connector may snap into the clasp. For example, the clasp may include one or more portions that define an opening for the connector to pass through the clasp; at least one of the portions of the clasp may be elastically deformed outward, as the connector passes through the opening, such as to allow a one-way passage of the connector (e.g., after the connector passes through the opening of the clasp, the deformed portion(s) may rebound back to the undeformed state, thereby securing the connector to the clasp).

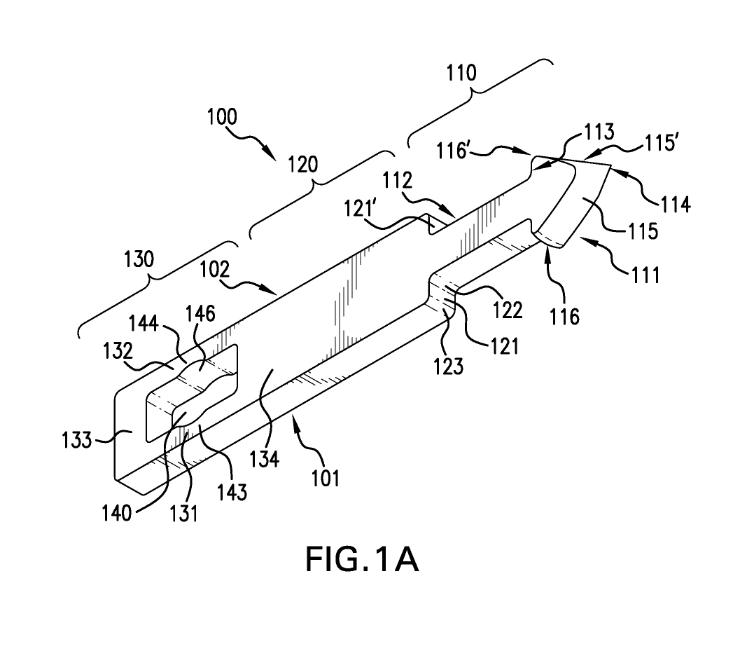

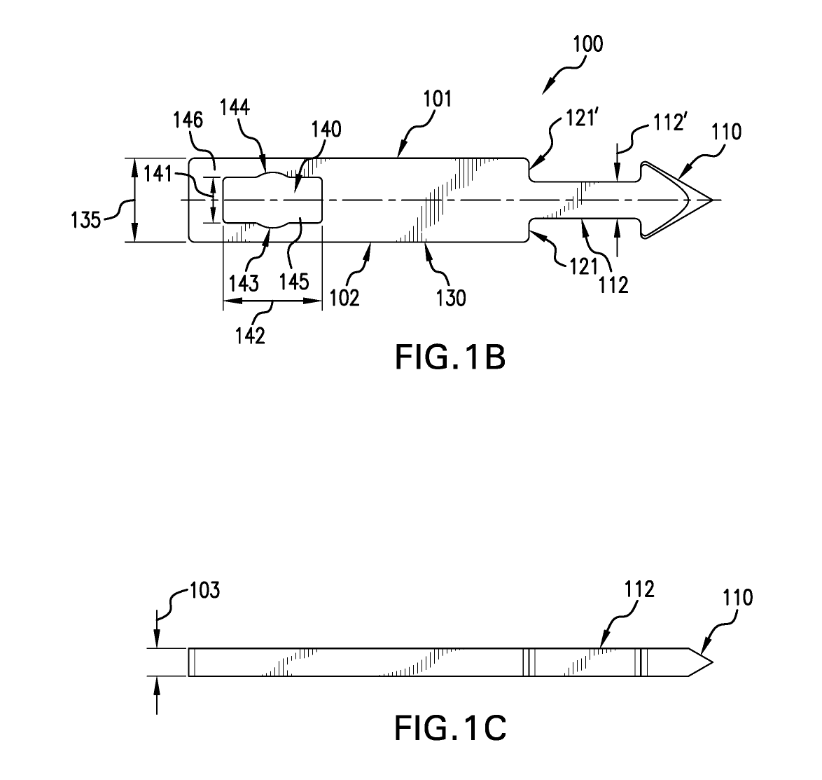

With reference to FIGS. 1A-1C, the suture 100 is shown flat or in an unfolded configuration (i.e., the suture 100 is shown in a generally two-dimensional or sheet-like configuration). As described below in more detail, the suture 100 may include shape-memory material and may be reconfigured into a different configuration.

Generally, the suture 100 includes a connector 110, a body 120, and a clasp 130. The body 120 may connect the connector 110 to the clasp 130. In the illustrated embodiment, the connector 110, body 120, and clasp 130 are integral with one another and can be made from a unitary piece of material. It should be appreciated, however, that the connector 110, body 120, and clasp 130 may be connected together with any number of suitable connections (e.g., welded connections, mechanical connections, etc.). In any event, the connector 110, body 120, and clasp 130 may be connected together in a manner that connector 110 can connect to and/or can be secured by the clasp 130 (e.g., as described below in more detail).

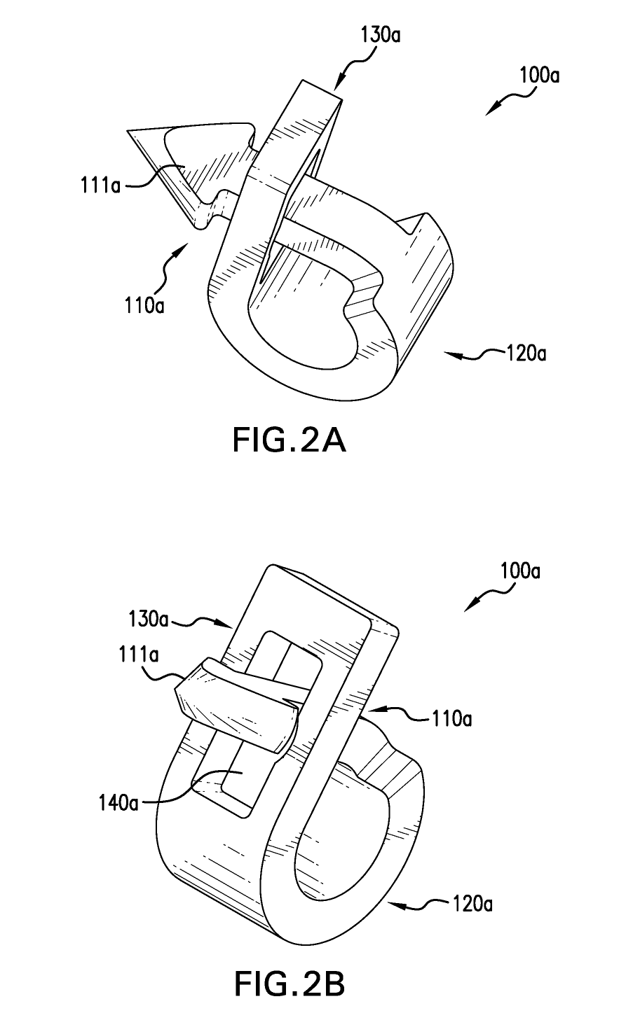

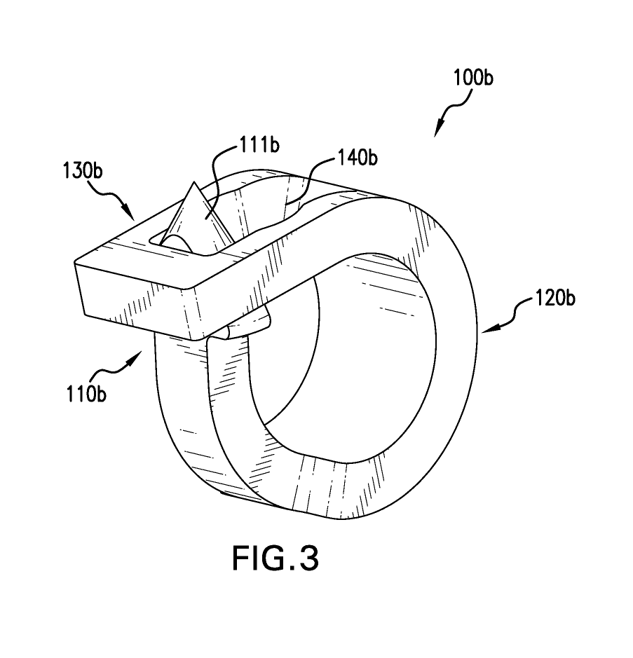

In some embodiments, the connector 110 and body 120 may include shape-memory materials, which may be similar to or the same as the material of the clasp 130. For example, any of the connector 110, body 120, or clasp 130 may include Nickel titanium (or Nitinol). The suture 100 may be heated to a selected temperature and held in a selected configuration to set the original or parent shape (i.e., the shape to which the suture 100 may return or would be biased toward). Generally, the suture 100 may be reconfigured using any suitable shape-setting method or process. For example, as described below (e.g., in connection with FIGS. 2A-3), the suture 100 may be bent in a manner that positions the connector 110 near and/or adjacent to the clasp 130 and may be held in such bent configuration at a selected or suitable temperature, in a manner that sets such configuration as the parent shape or the relaxed state of the suture 100. After the shape-setting process, when the suture 100 is biased away from the set shape to a loaded state, the suture 100 may be configured to return to the set shape, (e.g., releasing the suture 100 from a loaded state may return the suture 100 to the relaxed state and toward the set shape thereof).

With continued reference to FIGS. 1A-1C, the connector 110 may include a head 111 and a neck 112 connected together or integrally formed with one another. Generally, the connector 110 may be configured to pierce and/or penetrate one or more tissue portions (e.g., as the connector 110 moves toward the clasp 130). For example, the head 111 may be arrow-shaped or pointed. For example, the head 111 may include a base 113 and a tip 114 located on opposite sides of the head 111. The head 111 may include opposing surfaces tapering from the base 113 to the tip 114. The tip 114 may be suitably sharp (e.g., may be defined by a suitable small area at a location opposite to the base 113).

Moreover, the head 111 may include at least one curved surface and/or more than two sides tapering from the base 113 to the tip 114. In the illustrated embodiment, the tip 114 is formed by a cone that defines or forms the pointed tip 114; for example, at the meet-point of conical edge surfaces 115 and 115′ that extend from the base 113 (and away from the neck 112). It should be appreciated, however, that any suitable curved surface may extend from the base 113 and terminate at the tip 114 in a manner that forms the head 111 that is generally tapered. Furthermore, the side or edge surfaces may include or may be defined by multiple facets. For example, the edges of the head 111, extending from the base 113 to the tip 114, may be defined or formed by multiple surfaces (e.g., surfaces that may lie along or tangentially to or may approximate a curved surface, such as a conical surface).

The neck 112 is generally smaller or narrower than the base 113 (e.g., a transverse dimension of the base 113 may be greater than a transverse dimension of the neck 112). That is, opposing portions of the base 113 may extend from the neck 112 on opposing sides thereof (e.g., each of the opposing portions of the base 113 may extend away from the neck 112 by approximately the same distance, such that the portions of the base 113 are symmetrical about the neck 112). Moreover, the neck 112 may extend from the base 113 in a direction that is generally opposite to the tip 114.

In the illustrated embodiment of FIGS. 1A and 1B, the suture 100 may include outside radii or outside fillets 116, 116′ extending between the corresponding portions of the base 113 and the edge surfaces 115 and 115′. For example, the fillets 116, 116′ may be suitably sized to reduce or prevent snagging or unintentional cutting of tissue that may otherwise occur at shape edges or points. Moreover, the suture 100 may include inside radii or fillets extending between each of the opposing portions of the base 113 and the neck 112 (e.g., to reduce the stress concentration between the neck 112 and the base 113).

As mentioned above, the connector 110 may connect to the body 120. In the illustrated embodiment, the connector 110 and the body 120 are integrated with each other. For example, the body 120, head 111, and neck 112 may comprise the same material (e.g., the connector 110 and body 120 may be fabricated from a continuous sheet of material by stamping, laser cutting, etc., the continuous sheet). Alternatively, the connector 110 may be welded and/or otherwise attached to the body 120.

The neck 112 of the connector 110 may connect to the body 120. In the illustrated embodiment of FIGS. 1A and 1B, the body 120 is generally rectangular and has a continuous, generally linear edge (e.g., opposing edges of the body 120 define a generally uniform transverse dimension thereof). For example, the body 120 may have a longitudinal dimension that is greater than the transverse dimension thereof. It should be appreciated that, in some embodiments, the transverse dimension of the body 120 may be greater than the longitudinal dimension. Moreover, the body 120 may have any number of suitable shapes and/or sizes.

The width or transverse dimension of the body 120 may be greater than the width or transverse dimension of the neck 112. As mentioned above, the width or transverse dimension of the base 113 of the connector 110 may be greater than the width of the neck 112. Hence, for example, the neck 112 may form a stepped-down or reduced-width section of the suture 100 along the longitudinal direction thereof. In the illustrated embodiment, the width of the body 120 may be similar to or the same as the width of the base 113 (e.g., the body 120 and the connector 110 may be formed from a sheet material of the same width at the width of the body 120). Alternatively, the width of the body 120 may be greater or smaller than the width of the base 113. In at least one embodiment, the body 120 may form shoulders 121 and 121′ that extend outward from the neck 112 in a transverse direction.

The neck 112 may connect at an end of the body 120 (e.g., along a transverse dimension thereof) and may extend longitudinally therefrom. Hence, for example, the connector 110 and the body 120 may be longitudinally aligned with one another (e.g., when the suture 100 is in a generally planar or two-dimensional configuration).

In the illustrated embodiment of FIGS. 1A and 1B, the suture 100 includes inside radii or fillets 122, 122′ formed between each of the opposite sides or edges of the neck 112 and the respective shoulders 121, 121′ of the body 120. For example, the fillets 122, 122′ may reduce stress concentration in what otherwise may be sharp corners of the connection between the neck 112 and body 120. Alternatively, the neck 112 may connect to the body 120 in a manner that forms sharp corners at the connection therebetween. In the illustrated embodiment, the body 120 has outside radii or fillets 123, 123′ between the shoulder portions 121 and/or 121′ and lateral edges of the body 120.

As mentioned above, the head 111 of the connector 110 may be sized and shaped to penetrate and/or pierce one or more tissue portions. Tissue portions suitable for penetration and/or piercing by the head 111 of the connector 110 include animal tissue (e.g., human tissue, such as body lumen tissue, skin, muscle, etc.), synthetic tissue, thin sheets or film that may be connected to animal and/or synthetic tissue, and the like. Moreover, the suture 100 may connect together any number of suitable tissues in any number of suitable procedures. For example, the suture 100 may connect together two body lumens (e.g., in an anastomosis procedure).

The shoulders 121, 121′ may limit penetration of the suture 100 into the tissue; the fillets 123, 123′ between the shoulders 121, 121′ and the respective longitudinal edges may be configured to prevent unintentional cutting or tearing of the tissue that may otherwise occur at sharp corners. Furthermore, the shoulders 121 and/or 121′ may be sufficiently blunt and wide to prevent further penetration into the tissue past the neck 112. For this purpose also, the shoulders 121 and/or 121′ may have a slope with respect to the neck that is sufficiently shallow to prevent further penetration into the tissue. The shoulders 121, 121′may have a linear front surface facing the neck 112 and may be positioned generally perpendicular to the neck 112 or the longitudinal neck axis.

As described herein, the body 120 may have suitable width to prevent further penetration of the suture 100 into the tissue. For example, when the suture 100 is deployed, the tissue portions connected thereby may be positioned between the shoulders 121, 121′ of the body 120 and the base 113 of the connector 110 (e.g., along the neck 112 of the connector 110). The length or longitudinal dimension of the neck 112 may vary from one embodiment to another (e.g., the length of the neck 112 may be sized based on the thickness of the tissue portions intended to be secured together with the suture 100).

Again, the body 120 may be connected to or integrated with the clasp 130. In the illustrated embodiment of FIGS. 1A-1C, the body 120 and clasp 130 have the same width or transverse dimension. Moreover, the clasp 130 may have a generally constant or uniform width. For example, the suture 100 may have continuous edges 101 and 102 that may define the width of the body 120 and clasp 130. Alternatively, the width of the body 120 may be different from the width of the clasp 130 (e.g., the body 120 may be wider or narrower than the clasp 130). Moreover, the body 120 may be integrated or integrally formed with the clasp 130 or may be connected thereto (e.g., welded, mechanically attached, etc.).

One or more portions of the clasp 130 may define an opening that may be sized and configured to secure the connector 110 to the clasp 130. For example, portions 131, 132, 133, 134 of the clasp 130 may define an opening 140. The opening 140 may be sized and configured to secure the connector 110 to the clasp 130 (e.g., to secure the head 111 of the connector 110 to the clasp 130). In the illustrated embodiment of FIGS. 1A and 1B, the opening 140 has a transverse dimension 141 that is smaller than a longitudinal dimension 142 thereof (e.g., the opening 140 may have a generally or approximately rectangular shape). The opening 140 may have one or more fillets or inside radii at one or more corresponding corners of the opening 140.

The portions 131, 132, 133, 134 may include walls that define the opening 140. The walls may be suitably offset from the corresponding edges or outer periphery of the clasp 130 (e.g., the clasp 130 may have a width or transverse dimension 135 that may be suitably greater than the transverse dimension 141 of the opening 140). In an example, longitudinal walls 145, 146 that partially define the opening 140 may be generally linear or planar and may have corresponding notches 143, 144 therein. In the illustrated embodiment of FIGS. 1A and 1B, the notches 143, 144 are positioned about midway on each of the corresponding longitudinal walls 145, 146 of the opening 140. It should be appreciated, however, that the notches 143 and/or 144 may be positioned at any number of suitable locations along the longitudinal walls 145, 146 of the opening 140.

In the illustrated embodiment, the notches 143, 144 are curved or arcuate. Alternatively or additionally, at least one of the notches 143, 144 may be non-curved (e.g., V-shaped, rectangular, etc.). Generally, the notches 143, 144 may suitably reduce the strength and/or increase flexibility of the portions 131 and/or 132 (e.g., to facilitate passage of the connector 110 therethrough, as described below in more detail). For example, the notches 143, 144 extend into the surface of the longitudinal walls 145, 146 by suitable distance or depth (e.g., the distance between the outermost locations of the notches 143, 144 may be suitably greater than the transverse dimension 141 of the opening 140).

The transverse dimension 141 may be smaller than the width of the base 113 of the head 111 of the connector 110. For example, the portions 131 and/or 132 of the clasp 130 may elastically deform or bulge outward, to allow the head 111 to pass through the opening 140. Moreover, after the connector 110 passes through the opening 140, the clasp 130 may secure the head 111 (e.g., by preventing the connector 110 from exiting the opening 140). For example, after the head 111 passes through the opening 140 of the clasp 130, the portions 131, 132 of the clasp 130 may prevent the head 111 from exiting the opening 140. Moreover, connecting the connector 110 to the clasp 130 may position the head 111 on a first side of the clasp 130 and at least a portion of the neck 112 on an opposite side of the clasp 130. Furthermore, locking the connector 110 to the clasp 130 may secure together two or more tissue portions (as described below in more detail).

In one embodiment, the traverse dimension or distance between outermost portions or locations of the notches 143, 144 may be smaller than the width of the base 113 of the head 111. For example, all of the portions of the opening 140, including the notches 143 and 144, may be smaller or narrower than the base 113 of the head 111, such that head 111 may snap into the opening 140 (e.g., the opening 140 may be narrower than the base 113 of the head 111 along the entire length or longitudinal direction of the opening 140).

As mentioned above, the length of the neck 112 may vary from one embodiment to the next (e.g., depending on the thickness of the tissue portions intended for securing by the suture 100). Similarly, the overall length or longitudinal dimension of the suture 100 may vary from one embodiment to the next (e.g., any of the elements or components of the suture 100, such as the connector 110, body 120, or clasp 130, may have any suitable longitudinal length). Analogously, the suture 100 may have any number of suitable transverse dimensions (e.g., which may be similar to or the same as the transverse dimension 135 of the clasp 130). For example, the transverse dimension of the suture 100 may be in one or more of the following ranges: from 0.001 inches to 0.005 inches; from 0.004 inches to 0.010 inches; from 0.009 inches to 0.015 inches. It should be appreciated that the transverse dimension of the suture 100 may be outside of these ranges.

Moreover, the suture 100 may have any suitable thickness 103. For example, the thickness 103 of the suture 100 may be generally uniform for all of the portions of the suture 100. Alternatively, the thickness for the various portions of the suture 100 (e.g., for the connector 110, body 120, or clasp 130) may vary one from another. For example, the thickness 103 may be in one or more of the following ranges: from 0.001 inches to 0.003 inches; from 0.002 inches to 0.006 inches. The thickness also may be outside of these ranges. Furthermore, the thickness 103 may be smaller than the transverse dimension of the suture 100.

As described below in more detail, the suture 100 may be reconfigured or reshaped (e.g., heat-shaped) into one or more suitable shapes. The opening 140 may be suitably shaped to facilitate heat-shaping of the suture 100. For example, the opening 140 may be suitably elongated (e.g., sized along the longitudinal dimension of the suture 100) to facilitate heat-shaping of the suture 100 (e.g., the size and/or shape of the opening 140 may facilitate securing the head 110 to the clasp 130). For example, before heat-shaping, the suture 100 may be reconfigured to a closed or latched configuration, such that the head 110 is secured inside the opening 140 of the clasp 130; subsequently, the suture 100 may be heat treated to set the parent shape thereof (e.g., such that in the relaxed state, the suture 100 is configured to be in the latched configuration, with the head 110 interlocked or secured in the opening 140).

As mentioned above, the suture 100 may be reconfigured to have a different original or parent shape. FIGS. 2A-2B are side and front perspective views, respectively, of a suture 100a according to one or more embodiments. Generally, elements and components of the suture 100a may be similar to or the same as elements and components of the suture 100 (FIGS. 1A-1C). As described herein, suture 100a may be fabricated by reconfiguring (e.g., heat-shaping) the suture 100 (FIGS. 1A-1C). Moreover, in FIGS. 2A-2B, the suture 100a is shown in a latched position or configuration. For example, the suture 100a may be heat-shaped such that, in the relaxed or parent state, the suture 100a is in the latched or locked position.

For example, the suture 100a may include connector 110a, body 120a, and clasp 130a that may be similar to the connector 110, body 120, and clasp 130, respectively, of the suture 100 (FIGS. 1A-1C). In the illustrated embodiment of FIGS. 2A-2B, the suture 100a has a loop configuration (e.g., the suture 100 (FIGS. 1A-1C) may be reconfigured into the suture 100a in the manner described herein). Furthermore, when the suture 100a is in the parent shape or relaxed state (e.g., as shown in FIGS. 2A-2B), the connector 110a may be secured by the clasp 130a. Hence, for example, if the suture 100a is reconfigured into a loaded state or biased out of its parent shape (e.g., at room temperature), removing the biasing from the suture 100a may return the suture 100a to its parent shape (e.g., thereby connecting the connector 110a to the clasp 130a).

For example, the suture 100a may be reconfigured into a loaded state or biased out of its parent shape (e.g., such that the suture 100a may be ready for deployment). In some embodiments, a suture holder may releasably secure the suture 100a in the open configuration or loaded state. Moreover, when in the loaded state, the suture 100a may be positioned near or adjacent to one or more tissue portions and may be released in a manner that allows the suture 100a to reconfigure toward the parent shape or relaxed state (e.g., the suture 100a may be released from the suture holder and allowed to return to its parent shape or relaxed state).

For example, after being released from the loaded state, the suture 100a may penetrate the tissue portions and secure the connector 110a to the clasp 130a. As mentioned above, for example, the head 111a of the connector 110a may spread or enlarge the opening 140a of the clasp 130a (e.g., by elastically deforming the opening 140a) to pass therethrough; after the head 111a of the connector 110a passes through the opening 140a of the clasp 130a, the opening 140a may assume its un-deformed size and/or shape, thereby securing the connector 110a to the clasp 130a. Moreover, one or more of the portions of the clasp 130a that define the opening 140a may be suitably sized and/or shaped (e.g., may have suitable flexibility and/or resilience), such that the force applied to the walls of the opening 140a by the head 111a of the connector 110a is suitable for elastically deforming and/or temporarily enlarging the opening 140a to allow the head 111a of the connector 110a to pass therethrough.

When the suture 100a is in the latched position, one or more portions of the suture 100a may form a loop (e.g., looping about the tissue portions connected together by the suture 100a). Generally, the length and/or diameter (or arc) of the loop may vary from one embodiment to the next and may depend on the thickness of the tissue portions intended to be secured by the suture 100a.

In some embodiments, the suture may be configured such that in its parent shape or relaxed state at least one portion of the connector (e.g., the head of the connector) is positioned adjacent to the clasp, without securing the connector to the clasp. FIG. 3 is a front perspective view of a suture 100b according to some embodiments. Generally, elements and components of the suture 100b may be similar to or the same as elements and components of the suture 100 (FIGS. 1A-1C). With reference to FIG. 3, the suture 100b is shown in a pre-latched position. For example, the suture 100b may be heat-shaped such that, when the suture 100b is in the relaxed state or parent shape, the suture 100b is in the pre-latched position (e.g., such that further urging may be necessary to latch the suture 100b). For example, after deployment, the suture 100b may be squeezed to connect the head 111b with the clasp 130b thereof, such that the suture 100b is reconfigured from the pre-latched position to a closed or fully latched position (e.g., as shown in FIGS. 2A-2B). Alternatively, the suture 100b may have a relaxed state or parent shape where the suture 100b is fully latched (e.g., when the suture 100b is in the pre-latched position, as shown in FIG. 3, the suture 100b may be partially biased).

For example, the suture 100b may include connector 110b, body 120b, and clasp 130b that may be similar to the connector 110, body 120, clasp 130, respectively, of the suture 100 (FIGS. 1A-1C). In the illustrated embodiment of FIG. 3, the suture 100b has a loop-shaped configuration (e.g., the suture 100 (FIGS. 1A-1C) may be reconfigured into the suture 100b in the manner described herein). Furthermore, when the suture 100b is in the parent shape or relaxed state (e.g., as shown in FIG. 3), the connector 110b may be positioned adjacent to the clasp 130b but remain unconnected therewith or unsecured thereby. Hence, for example, if the suture 100b is reconfigured into a loaded state or biased out of its parent shape (e.g., at room temperature), removal of the biasing of the suture 100b may return the suture 100b to its parent shape, positioning the connector 110b adjacent to the clasp 130b.

In the illustrated embodiment, the head 111b of the connector 110b is positioned adjacent to the clasp 130b when the suture 100b is in the parent shape or relaxed state. For example, the tip of the head 111b of the connector 110b may be positioned in the opening 140b of the clasp 130b. To close the suture 100b, the head 111b of the connector 110b may be forced through the opening 140b in the clasp 130b to snap the connector 110b to the clasp 130b.

In the illustrated embodiment, closing the suture 100b to secure the connector 110b to the clasp 130b reconfigures or biases the suture 100b from its parent shape or relaxed state. As such, when the suture 100b is in a closed configuration (e.g., when the head 111b of the connector 110b has passed through the hole in the clasp 130b), the suture 100b may bias the connector 110b away from the clasp 130b. Hence, for example, the base of the head 111b of the connector 110b may be pressed against one or more portions of the clasp 130b when the suture 100b is in the closed configuration. For example, such biasing may prevent excessive compression of the tissue portions by the suture 100b after the tissue portions are secured by the suture 100b.

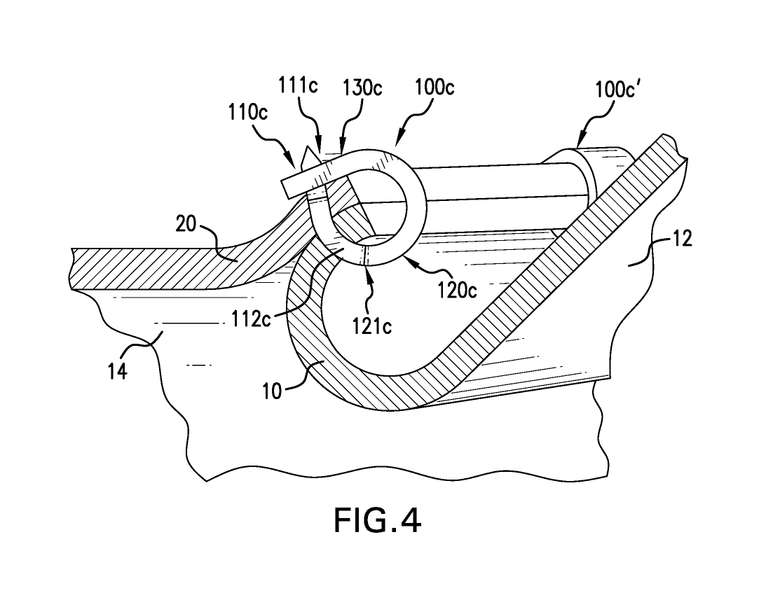

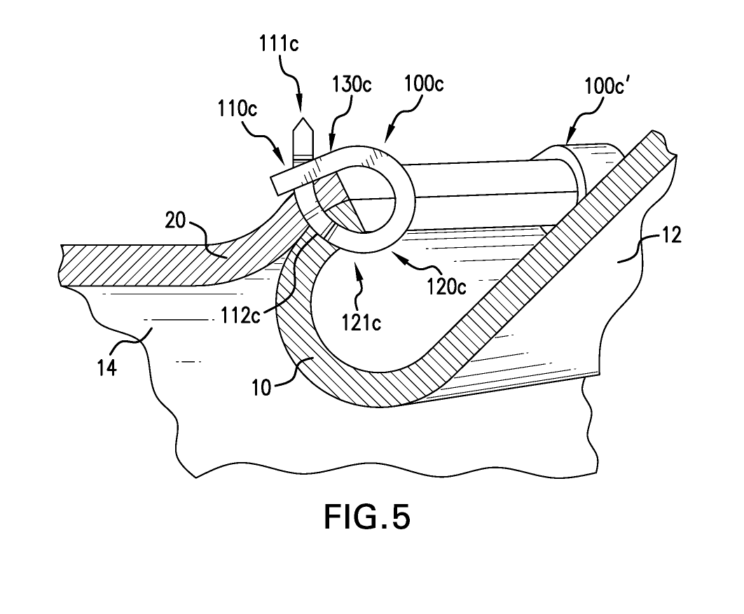

FIGS. 4 and 5 are side, perspective views of a suture 100c holding together tissue portions 10 and 20. In the depicted embodiment, the tissue portions 10, 20 are positioned such that a lumen 12 adjacent one portion 10 will be fluidly connected to a lumen 14 adjacent the other portion 20 when the portions 10, 20 are connected. FIG. 4 shows the suture 100c in a pre-latched position and FIG. 5 shows the suture 100c in a latched or secured position. Generally, elements and components of the suture 100c may be similar to or the same as elements and components of any of the sutures described herein. For example, the suture 100c may include connector 110c, body 120c, and clasp 130c that may be similar to the connector 110b, body 120b, and clasp 130b, respectively, of the suture 100b (FIG. 3).

For example, when the suture 100c is deployed, the clasp 130c may be positioned on one side of stacked of tissue portions, and at least a portion of the suture 100c may be positioned on another side (e.g., the suture 100c may loop about the tissue portions 10 and 20). As shown in FIGS. 4 and 5, head 111c of the connector 110c may be positioned on one side of the clasp 130c, while neck 112c of the connector 110c may be positioned on another side of the clasp 130c. Moreover, the neck 112c may extend through the tissue portion 10 and/or through the tissue portion 20.

As described above, the suture 100c may include shoulders, such as a shoulder 121c that may be formed at the intersection between the neck 112c and the body 120c. The shoulder 121c may be configured to limit penetration of the suture 100c in the tissue portions 10, 20 (e.g., the shoulder 121c may stop the suture 100c from further penetrating the tissue portions 10, 20). Moreover, the suture 100c may be suitably sized and/or curved, such that after deployment, the tissue portions 10 and 20 are suitably compressed and/or secured together between the shoulder 121c and the clasp 130c.

The suture 100c may be moved from a pre-latched position as shown in FIG. 4 to a latched position as shown in FIG. 5 by moving the head 111c through the opening 140 (see FIGS. 1A and 1B) until the base 113 (see FIGS. 1A and 1B) of the head 111c has passed through the opening 140. The head 111c may be moved such as by squeezing the clasp 130c and neck 112c towards each other with any suitable tool, such as forceps.

It should be appreciated that any number of sutures may be deployed to secure together two or more tissue portions. For example, in addition to the suture 100c, the suture 100c′ may secure the tissue portions 10 and 20. Moreover, the sutures, such as suture 100c and suture 100c′ may be suitably spaced one from another, to facilitate suitable contact and/or connection between the tissue portions 10, 20 secured thereby.

While the foregoing is directed to certain embodiments, other and further embodiments may be devised without departing from the basic scope thereof, and the scope thereof is determined by the claims that follow.

While various aspects and embodiments have been disclosed herein, other aspects and embodiments are contemplated. The various aspects and embodiments disclosed herein are for purposes of illustration and are not intended to be limiting. Additionally, the words “including,” “having,” and variants thereof (e.g., “includes” and “has”) as used herein, including the claims, shall be open ended and have the same meaning as the word “comprising” and variants thereof (e.g., “comprise” and “comprises”).

What is claimed is:

1. A suture, comprising:
a connector that includes:
a neck, and
a head having a tip and a base having a width that is wider than the neck in a transverse direction, the head tapering from the base to the tip such that the head is configured to pierce animal tissue;
a clasp including sheet material that defines an opening that, in a transverse direction, is narrower than the width of the base of the head, the clasp being resilient to allow the opening to elastically expand in the transverse direction upon passing the head therethrough and contract about the neck to secure the connector to the clasp; and
a body connecting the connector to the clasp, the body being made of a shape-memory material.

2. The suture of claim 1, wherein in a relaxed state, the body and a portion of the neck are positioned on one side of the clasp and the head is positioned on an opposite side of the neck such that the head is secured to the clasp.

3. The suture of claim 1, wherein in a relaxed state, the body generally aligns the tip with the clasp opening.

4. The suture of claim 1, wherein in a relaxed state, the tip is positioned adjacent to the clasp.

5. The suture of claim 1, wherein the head has at least one curved surface or more than two sides tapering from the base to the tip.

6. The suture of claim 1, wherein the body includes shoulders that extend outward from opposites sides of the neck in a transverse direction.

7. The suture of claim 6, wherein the shoulders are positioned generally perpendicularly to a longitudinal axis of the neck and are configured to prevent penetration of the suture into tissue past the neck of the connector.

8. The suture of claim 6, wherein a fillet is positioned between each of the opposite sides of the neck and the respective shoulder.

9. The suture of claim 6, wherein a fillet is positioned between each shoulder and a respective lateral edge of the body.

10. The suture of claim 1, wherein the opening is partially defined by longitudinal walls having notches formed therein.

11. A suture, comprising:
a connector that includes:
a neck, and
a head connected to the neck, the head including:
a tip, and
a base having a width that is wider than the neck in a transverse direction, the head tapering from the base to the tip such that the head is configured to pierce animal tissue;
a clasp including sheet material that defines an opening that is sized and configured to secure the connector to the clasp;
one or more edge-stops connected to the neck and spaced from the base of the head, the one or more edge-stops together with the base of the head defining a tissue-capture portion of the neck; and
a body connecting the connector to the clasp, the body being made of a shape-memory material.

12. The suture of claim 11, wherein in a relaxed state, the body and a portion of the neck are positioned on one side of the clasp and the head is positioned on an opposite side of the neck such that the head is secured to the clasp.

13. The suture of claim 11, wherein in a relaxed state, the body generally aligns the tip with the clasp opening.

14. The suture of claim 11, wherein in a relaxed state, the tip is positioned adjacent to the clasp.

15. The suture of claim 11, wherein the edge-stops are positioned generally perpendicularly to a longitudinal axis of the neck and are configured to prevent penetration of the suture into tissue past the neck of the connector.

16. A method of connecting two or more tissue portions, the method comprising:
positioning a suture adjacent to at least one of the two or more tissue portions, the suture being in a loaded state and including:
a connector that includes:
a neck, and
a head having a tip and a base having a width that is wider than the neck in a transverse direction, the head tapering from the base to the tip such that the head is configured to pierce animal tissue;
a clasp including sheet material that defines an opening that, in a transverse direction, is narrower than the width of the base of the head, the clasp being resilient to allow the opening to elastically expand in the transverse direction upon passing the head therethrough and contract about the neck to secure the connector to the clasp;
a body connecting the connector to the clasp; and
releasing the suture from the loaded state, and thereby piercing the two or more tissue portions with the piercing tip of the connector.

17. The method of claim 16, wherein releasing the suture from the loaded state positions the connector adjacent to the clasp.

18. The method of claim 16, wherein releasing the suture from the loaded state secures the connector to the clasp.

19. The method of claim 16, further comprising securing the connector to the clasp.

20. The method of claim 16, wherein the body includes shoulders that extend outward from opposites sides of the neck in a transverse direction and, after releasing the suture from the loaded state, the suture is positioned such that the tissue portions are positioned between the shoulders of the body and the base of the connector.
